(19) United States
(12) Patent Application Publication
(10) Pub. No.: US 2022/0266051 A1
(43) Pub. Date: Aug. 25, 2022

(54) WEARABLE BIOPHOTONIC SYSTEMS

(71) Applicant: KLOX TECHNOLOGIES INC., Laval (CA)

(72) Inventors: Lise HEBERT, Montreal (CA); Francesco BELLINI, Calgary (CA); Shannon CAMPBELL, Montreal (CA)

(21) Appl. No.: 17/625,493

(22) PCT Filed: Jul. 7, 2020
(86) PCT No.: PCT/CA2020/050941

§ 371 Date: Jan. 7, 2022

Related U.S. Application Data
(60) Provisional application No. 62/871,558, filed on Jul. 8, 2019.

(51) Int. Cl. A61N 5/06; C09K 11/02; C09K 11/06; C09B 11/00; C09B 67/20

(52) U.S. Cl. CPC A61N 5/062; C09K 11/02; C09K 11/06; C09B 11/00; C09B 67/0063; A61N 2005/0652; C09K 2211/1018

(57) ABSTRACT

The present technology relates to a biophotonic system comprising at least one chromofilm; and a light-emitting support operably connectable to the chromofilm. Operable connection of the light-emitting support and the at least one chromofilm allows for the light-emitting support to illuminate the at least one chromofilm.

### CROSS-REFERENCE TO RELATED APPLICATION

This application claims the benefit of and priority to U.S. provisional patent application No. 62/871,558, filed on Jul. 8, 2019; the content of all of which is herein incorporated in entirety by reference.

### FIELD OF TECHNOLOGY

The present disclosure generally relates to wearable biophotonic systems as well as to their use in biophotonic therapy of subjects.

### BACKGROUND INFORMATION

Phototherapy has recently been recognized as having wide range of applications in both the medical and cosmetic fields including use in surgery, therapy and diagnostics. For example, phototherapy has been used to treat cancers and tumors with lessened invasiveness, to disinfect target sites as an antimicrobial treatment, to promote wound healing, and for facial skin rejuvenation.

Phototherapy involves the application of a photosensitive agent to target tissue then exposing the target tissue to a light source after a determined period of time during which the photosensitizer is absorbed by the target tissue. Such regimens, however, are often associated with undesired side-effects, including systemic or localized toxicity to the patient or damage to non-targeted tissue. Moreover, such existing regimens often demonstrate low therapeutic efficacy due to, for example, the poor selectivity of the photosensitive agents into the target tissues.

Therefore, it is an object of the present disclosure to provide new and improved biophotonic systems, in particular new and improved wearable biophonic systems for use in biophotonic therapy.

### SUMMARY OF TECHNOLOGY

According to various aspects, the present technology relates to a biophotonic system comprising: at least one chromofilm; and a light-emitting support operably connectable to the chromofilm; wherein operable connection of the light-emitting material and the at least one chromofilm allows for photoactivation of the at least one chromofilm. In various implementations of these aspects, the chromofilm comprise light-absorbing agents. In various implementations of these aspects, the light-emitting support comprises a flat support and a light-emitting system. In some instances, the light-emitting elements are LEDs lights. In some other implementations, the light-emitting elements are quantum dots.

According to various aspects, the present technology relates to methods for biophotonic treatment of a tissue or skin area of a subject, the method comprising applying at least one chromofilm to the area of the subject to be treated, then placing the light-emitting support on the chromofilm in an operable configuration, and activating the light-emitting support to illuminate the chromofilm. In some implementations, following the illumination, the light-emitting support is removed from the chromofilm, while the chromofilm is maintained on the area of the subject.

According to various aspects, the present technology relates to a kit for biophotonic treatment. The kit comprises the biophotonic system as defined herein.

According to various aspects, the present technology relates to a kit for biophotonic treatment. The kit comprises at least one chromofilm; a light-emitting support operably connectable to the chromofilm; wherein operable connection of the light-emitting support and the at least one chromofilm allows for photoactivation of the at least one chromofilm.

### BRIEF DESCRIPTION OF THE DRAWINGS

All features of embodiments which are described in this disclosure are not mutually exclusive and can be combined with one another. For example, elements of one embodiment can be utilized in the other embodiments without further mention. A detailed description of specific embodiments is provided herein below with reference to the accompanying drawings in which:

FIG. 1 is a schematic representation of a biophotonic system according to one embodiment of the present technology wherein the chromofilm and the light-emitting support are not in an operational configuration.

### DETAILED DESCRIPTION OF TECHNOLOGY

Before continuing to describe the present disclosure in further detail, it is to be understood that this disclosure is not limited to specific compositions or process steps, as such may vary. It must be noted that, as used in this specification and the appended embodiments, the singular form “a”, “an” and “the” include plural referents unless the context clearly dictates otherwise.

As used herein, the term “about” in the context of a given value or range refers to a value or range that is within 20%, within 10%, and more within 5% of the given value or range.

It is convenient to point out here that “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The term “biophotonic” as used herein refers to the generation, manipulation, detection and application of photons in a biologically relevant context. In other words, compositions exert their physiological effects primarily due to the generation and manipulation of photons.

The term “actinic light” is intended to mean light energy emitted from a specific light source (lamp, LED, or laser) and capable of being absorbed by matter (e.g. the light-absorbing molecule defined below) and produce an identifiable or measurable change when it interacts with it; as clinically identifiable change we can presume a change in the color of the light-absorbing molecule used (e.g. from red to transparent).

The term “topical” means as applied to body surfaces, such as the skin, mucous membranes, vagina, oral cavity, internal surgical wound sites, and the like.

The term “light-absorbing agent” refers to a compound which, when illuminated by light irradiation, is capable of absorbing the light.

The term “time of illumination to actinic light” is intended to mean the time a tissue, skin or wound is illuminated with actinic light per application of actinic light. The term “total time of illumination to actinic light” is intended to mean the cumulative time a tissue, skin or wound is illuminated with actinic light after several application of actinic light.

“Wound” means an injury to any tissue, including for example, acute, subacute, and non-healing wounds. Examples of wounds may include both open and closed wounds. Wounds include, for example, skin diseases that result in a break of the skin or in a wound, clinically infected wounds, burns, early stage burns, incisions, excisions, lesions, lacerations, abrasions, puncture or penetrating wounds, gunshot wounds, surgical wounds, contusions, hematomas, crushing injuries, ulcers, scarring (cosmesis), wounds caused by periodontitis. As used herein, the term “wounds” also includes “non-healing wounds” and “chronic wounds”. “Non-healing wounds” means wounds that do not heal in an orderly set of stages and a predictable amount of time and rate in the way that most normally-healing wounds heal, and non-healing wounds include, but are not limited to: incompletely healed wounds, delayed healing wounds, impaired wounds, difficult to heal wounds and chronic wounds. Examples of such non-healing wounds include but are not limited to: diabetic foot ulcers, vasculitic ulcers, pressure ulcers, decubitus ulcers, infectious ulcers, trauma-induced ulcers, burn ulcers, ulcerations associated with pyoderma gangrenosum, dehiscent and mixed ulcers. A non-healing wound may include, for example, a wound that is characterized at least in part by 1) a prolonged inflammatory phase, 2) a slow forming extracellular matrix, and/or 3) a decreased rate of epithelialization or closure. “Chronic wound” is a wound that does not heal in an orderly set of stages and in a predictable amount of time the way most wounds do; wounds that do not heal within three months are often considered chronic. Chronic wounds include venous ulcers, venous stasis ulcers, arterial ulcers, pressure ulcers, diabetic ulcers, and diabetic foot ulcers.

In one embodiment, the present disclosure relates to a biophotonic system which may be used to treat a subject, in particular to treat a tissue or a skin area of a subject. In some instances, the subject to be treated by the biophotonic system of the present disclosure is a human or an animal. The biophotonic system of the present technology comprises at least two components. One component of the biophotonic system of the present disclosure comprises light-absorbing agents. Another component of the biophotonic system of the present disclosure provides light source. The biophotonic system operates when the two components are positioned such that light emitted from the light source of one component photoactivates the light-absorbing agents of the other component. Photoactivation of the light-absorbing agents causes the light-absorbing agents and/or the component comprising the light-absorbing agents to emit fluorescence.

In one embodiment, the present disclosure relates to a biophotonic system which may be used in methods for treating a subject. The biophotonic system comprises a chromofilm and a light-emitting support. The chromofilm and the light-emitting support connect together in an operative manner in which the light emitted by the light-emitting support photoactivates the chromofilm. In some instances, the photoactivated chromofilm emits fluorescence. The chromofilm comprises light-absorbing molecules uniformly or non-uniformly distributed throughout the film.

In some implementations of these embodiments, the light-emitting support comprises a plurality of light-emitting elements. The light-emitting elements may be uniformly or non-uniformly distributed throughout the substrate. In some implementations, the distribution of the light-absorbing agents in the chromofilm coincides with the distribution of the light-emitting elements on the light-emitting support when the chromofilm and the light-emitting support are in an operational configuration. In the operational configuration, the light emitted by the plurality of light emitting elements photoactivates the light-absorbing agents in the chromofilm. In some implementations of these embodiments, the chromofilm and the light-emitting support have a shape that is complementary to one another to allow operational configuration.

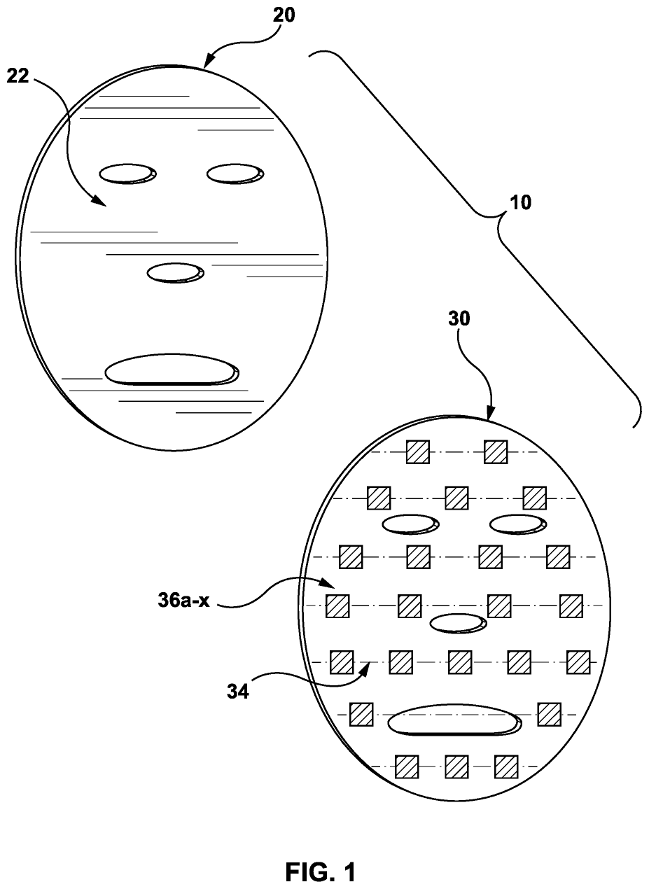

FIG. 1 depicts a biophotonic system according to one embodiment of the present technology. In this embodiment, the biophotonic system (10) comprises a chromofilm (20) and a light-emitting support (30).

In this embodiment, the chromofilm (20) comprises a silicone-based film (22) that is substantially flat and shaped into a mask for facial application. The chromofilm (20) comprises light-absorbing agents. In use, the chromofilm (20) is suitable to be placed on the tissue or skin of a subject. In some implementations of this embodiment, the silicone-based film may be replaced by any suitable copolymer material such as, but not limited to, copolymers of tetrafluoroethylene; 2,2-bis(trifluoromethyl)-4,5-difluoro-1,3-dioxole, polytetrafluoroethylene (PTFE) and fluorinated ethylene propylene.

The light-emitting support (30) comprises a support material (32) and a light-emitting system (34). The light-emitting system (34) comprises a plurality of light-emitting elements (36ₐ₋ₓ). In this embodiment, the support material (32) has a shape that is similar to the shape of the chromofilm (20) so that the light-emitting support (30) may be placed onto the chromofilm (30) to illuminate the chromofilm (20).

In the configuration depicted in FIG. 1, the chromofilm (20) and the light-emitting support (30) are separated from each other and hence are not in an operational configuration. However, in operational configuration, the light-emitting support (30) is placed in operational vicinity with the chromofilm (20) (e.g., placed on top of the chromofilm 20) such that the plurality of light-emitting elements (36ₐ₋ₓ) illuminates the light-absorbing agents of the chromofilm (20).

In some embodiments, the light-emitting support (30) is connected to the chromofilm (20) with use of fasteners (e.g., clips, buttons, adhesives, or the like). In some other embodiments, the shape of the light-emitting support (30) is complementary to the shape of the chromofilm (20) and this complementarity in shape maintains the chromofilm (20) and light-emitting support (30) connected to each other.

In some embodiments, the biophotonic system may comprise more than one chromofilm and/or more than one light-emitting support. A light-emitting support may be used with a combination of chromofilms each comprising different light-absorbing agents. The chromofilms may be stacked on top of one another to provide a combination of light-absorbing agents.

i) Chromofilm

In some embodiments, the chromofilm comprises a silicone-based film having light-absorbing agents distributed therein. The silicone-based film comprises a silicone phase and a surfactant phase, with the light-absorbing agents being solubilized in the surfactant phase. In some embodiments, the surfactant phase is emulsified in the silicone phase. The light-absorbing agents in the silicone-based biophotonic composition may be photoactivated by light. This activation accelerates the dispersion of light energy, leading to light carrying on a therapeutic effect on its own, and/or to the photochemical activation of other agents contained in the film. This may lead to the breakdown of the light-absorbing agents and, in some embodiments, ensure that the silicone-based film is for single-use.

When a light-absorbing agent absorbs a photon of a certain wavelength, it becomes excited. This is an unstable condition and the molecule tries to return to the ground state, giving away the excess energy. For some light-absorbing agents, it is favorable to emit the excess energy as light when returning to the ground state. This process is called fluorescence. The peak wavelength of the emitted fluorescence is shifted towards longer wavelengths compared to the absorption wavelengths due to loss of energy in the conversion process. Without being bound to theory, it is thought that fluorescent light emitted by photoactivated light-absorbing agents may have therapeutic properties due to its femto-, pico-, or nano-second emission properties which may be recognized by biological cells and tissues, leading to favourable biomodulation. Furthermore, generally, the emitted fluorescent light has a longer wavelength and hence a deeper penetration into the tissue than the activating light. Irradiating tissue with such a broad range of wavelength, including in some embodiments the activating light which passes through the chromofilm, may have different and complementary effects on the cells and tissues. In other words, light-absorbing agents are used in the chromofilm of the present disclosure for therapeutic effect on tissues. This is a distinct application of these light-absorbing agents and differs from the use of light-absorbing agents as simple stains or as catalysts for photo-polymerization.

The chromofilms of the present disclosure may have topical uses such as a mask or a wound dressing. In some embodiments, the chromofilms are cohesive. The cohesive nature of these chromofilms may provide ease of removal from the site of treatment and hence provide for a convenient ease of use.

Suitable light-absorbing agents that may be present in the chromofilms of the present technology can be fluorescent compounds (or stains) (also known as “fluorochromes” or “fluorophores”). In some instances, the light-absorbing agent is a naturally-occurring light-absorbing agent. In some instances, the light-absorbing agent is a synthetic light-absorbing agent.

Light-absorbing agents which are not well tolerated by the skin or other tissues can be included in the chromofilms of the present disclosure, as in certain embodiments, the light-absorbing agents are encapsulated within the surfactant phase of the emulsion in the silicone continuous phase.

In some embodiments, the light-absorbing agent absorbs at a wavelength in the range of the visible spectrum, such as at a wavelength of about 380-800 nm, 380-700, 400-800, or 380-600 nm. In other embodiments, the light-absorbing agent absorbs at a wavelength of about 200-800 nm, 200-700 nm, 200-600 nm or 200-500 nm. In one embodiment, the light-absorbing agent absorbs at a wavelength of about 200-600 nm. In some embodiments, the light-absorbing agent absorbs light at a wavelength of about 200-300 nm, 250-350 nm, 300-400 nm, 350-450 nm, 400-500 nm, 450-650 nm, 600-700 nm, 650-750 nm or 700-800 nm. It will be appreciated to those skilled in the art that optical properties of a particular light-absorbing agent may vary depending on the light-absorbing agent's surrounding medium. Therefore, as used herein, a particular light-absorbing agent's absorption and/or emission wavelength (or spectrum) corresponds to the wavelengths (or spectrum) measured in a chromofilm of the present disclosure.

The chromofilm disclosed herein may include at least one additional light-absorbing agent or second light-absorbing agent. Combining light-absorbing agents may increase photo-absorption by the combined dye molecules and enhance absorption and photo-biomodulation selectivity. This creates multiple possibilities of generating new photosensitive mixtures. Thus, in certain embodiments, chromofilms of the disclosure include more than one light-absorbing agent, and when illuminated with light, energy transfer can occur between the light-absorbing agents. This process, known as resonance energy transfer, is a widely prevalent photophysical process through which an excited ‘donor’ light-absorbing agent (also referred to herein as first light-absorbing agent) transfers its excitation energy to an ‘acceptor’ light-absorbing agent (also referred to herein as second light-absorbing agent). The efficiency and directedness of resonance energy transfer depends on the spectral features of donor and acceptor light-absorbing agents. In particular, the flow of energy between light-absorbing agents is dependent on a spectral overlap reflecting the relative positioning and shapes of the absorption and emission spectra. More specifically, for energy transfer to occur, the emission spectrum of the donor light-absorbing agent must overlap with the absorption spectrum of the acceptor light-absorbing agent.

The light-absorbing agent may be present in an amount of about 0.001-40% per weight of the silicone-based film or of the surfactant phase. In certain embodiments, the light-absorbing agent is present in an amount of about 0.001-3%, 0.001-0.01%, 0.005-0.1%, 0.1-0.5%, 0.5-2%, 1-5%, 2.5-7.5%, 5-10%, 7.5-12.5%, 10-15%, 12.5-17.5%, 15-20%, 17.5-22.5%, 20-25%, 22.5-27.5%, 25-30%, 27.5-32.5%, 30-35%, 32.5-37.5%, or 35-40% per weight of the silicone-based film or of the surfactant phase.

The concentration of the light-absorbing agent to be used can be selected based on the desired intensity and duration of the biophotonic activity from the chromofilm, and on the desired medical or cosmetic effect. For example, some dyes such as xanthene dyes reach a ‘saturation concentration’ after which further increases in concentration do not provide substantially higher emitted fluorescence. Further increasing the light-absorbing agent concentration above the saturation concentration can reduce the amount of activating light passing through the matrix. Therefore, if more fluorescence is required for a certain application than activating light, a high concentration of light-absorbing agent can be used. However, if a balance is required between the emitted fluorescence and the activating light, a concentration close to or lower than the saturation concentration can be chosen.

Suitable light-absorbing agents that may be used in the silicone-based biophotonic compositions of the present disclosure include, but are not limited to the following: chlorophyll dyes, xanthene dyes [examples of xanthene dyes include, but are not limited to, eosin B, eosin B (4′,5′-dibromo,2′,7′-dinitr-o-fluorescein, dianion); Eosin Y; eosin Y (2′,4′,5′,7′-tetrabromo-fluoresc-ein, dianion); eosin (2′,4′,5′,7′-tetrabromo-fluorescein, dianion); eosin (2′,4′,5′,7′-tetrabromo-fluorescein, dianion) methyl ester; eosin (2′,4′,5′,7′-tetrabromo-fluorescein, monoanion) p-isopropylbenzyl ester; eosin derivative (2′,7′-dibromo-fluorescein, dianion); eosin derivative (4′,5′-dibromo-fluorescein, dianion); eosin derivative (2′,7′-dichloro-fluorescein, dianion); eosin derivative (4′,5′-dichloro-fluorescein, dianion); eosin derivative (2′,7′-diiodo-fluorescein, dianion); eosin derivative (4′,5′-diiodo-fluorescein, dianion); eosin derivative (tribromo-fluorescein, dianion); eosin derivative (2′,4′,5′,7′-tetrachlor-o-fluorescein, dianion); eosin; eosin dicetylpyridinium chloride ion pair; erythrosin B (2′,4′,5′,7′-tetraiodo-fluorescein, dianion); erythrosin; erythrosin dianion; erythiosin B; fluorescein; fluorescein dianion; phloxin B (2′,4′,5′,7′-tetrabromo-3,4,5,6-tetrachloro-fluorescein, dianion); phloxin B (tetrachloro-tetrabromo-fluorescein); phloxine B; rose bengal (3,4,5,6-tetrachloro-2′,4′,5′,7′-tetraiodofluorescein, dianion); pyronin G, pyronin J, pyronin Y; Rhodamine dyes such as rhodamines include 4,5-dibromo-rhodamine methyl ester; 4,5-dibromo-rhodamine n-butyl ester; rhodamine 101 methyl ester; rhodamine 123; rhodamine 6G; rhodamine 6G hexyl ester; tetrabromo-rhodamine 123; and tetramethyl-rhodamine ethyl ester], methylene blue dyes, azo dyes, and natural dyes.

In certain embodiments, the silicone-based biophotonic compositions of the present disclosure includes any of the light-absorbing agents listed above, or a combination thereof, so as to provide a synergistic biophotonic effect at the application site.

In some embodiments, the composition includes Eosin Y as a first light-absorbing agent and any one or more of Rose Bengal, Fluorescein, Erythrosine, Phloxine B, chlorophyll as a second light-absorbing agent. It is believed that these combinations have a synergistic effect as they can transfer energy to one another when activated due in part to overlaps or close proximity of their absorption and emission spectra. This absorbed and re-emitted light is thought to be transmitted throughout the composition, and also to be transmitted into the site of treatment.

In further embodiments, the silicone-based biophotonic composition may include, for example, the following synergistic combinations: Eosin Y and Fluorescein; Fluorescein and Rose Bengal; Erythrosine in combination with Eosin Y, Rose Bengal or Fluorescein; Phloxine B in combination with one or more of Eosin Y, Rose Bengal, Fluorescein and Erythrosine.

In some embodiments, the light-absorbing agent or light-absorbing agents are selected such that their emitted fluorescent light, on photoactivation, is within one or more of the green, yellow, orange, red and infrared portions of the electromagnetic spectrum, for example having a peak wavelength within the range of about 490 nm to about 800 nm. In certain embodiments, the emitted fluorescent light has a power density of between 0.005 to about 10 mW/cm², about 0.5 to about 5 mW/cm².

In some embodiments, the chromofilm comprises a plurality of pores. In some instances, the pores facilitate the absorption and the emission of light in and from the chromofilm. In some other instances, the plurality of pores facilitates the treatment of a subject by for example, allowing exudates to evacuate the chromofilm.

In some implementations of these embodiments, the pores have an average size of between about 1 μm and about 5 mm. In some embodiments, the pores have a size that allows passage of air, gas and/or fluid through the chromofilm.

In some implementations of these embodiments, the plurality of pores are formed during polymerization of the silicone-base film by, for example, pouring the liquid silicone-based film into which the light-absorbing agent has been integrated onto a mold (e.g., grid, mesh, or the like) that forms pores into the silicone-base film during polymerization and solidification.

In some embodiments, light-absorbing agents of the present technology are sprayed onto a silicone-base film in replacement or in addition to the light-absorbing agents distributed throughout the silicone-base film.

In some instances, the chromofilm of the present disclosure comprises silica to facilitate and/or to improve reflection of light within the chromofilm.

The chromofilm of the present disclosure may comprise a surfactant phase. The surfactant may be present in an amount of at least 5%, 10%, 15%, 20%, 25%, or 30% of the chromofilm. In certain embodiments, the surfactant phase comprises a block copolymer. The term “block copolymer” as used herein refers to a copolymer comprised of 2 or more blocks (or segments) of different homopolymers. The term homopolymer refers to a polymer comprised of a single monomer. In certain embodiments of any of the foregoing or following the block copolymer is biocompatible. A polymer is “biocompatible” in that the polymer and degradation products thereof are substantially non-toxic to cells or organisms, including non-carcinogenic and non-immunogenic, and are cleared or otherwise degraded m a biological system, such as an organism (patient) without substantial toxic effect. In certain embodiments of the disclosure the surfactant phase comprises polylactic acid (PLA), or polyglycolic acid (PGA) or poly(lactic-co-glycolic acid) (PLGA) or polycaprolactone (PCL) or polydioxanone (PDO). Surfactants may be selected according to effects they will have on the optical transparency of the biophotonic membrane. The silicone-based biophotonic composition should be able to transmit sufficient light to illuminate the at least one chromophore and, in embodiments where fluorescence is emitted by the photoactivated chromophore, the surfactant phase should also be able to transmit the emitted fluorescent light to tissues.

In one embodiment, the chromofilms of the present disclosure comprise a continuous phase of silicone. Silicones are synthetic polymers containing chains consisting of (—Si—O—) repeating unit with two organic groups attached directly to the Si atom. In certain embodiments, the silicone is a polydimethylsiloxane (PDMS) fluid (Me₂SiO)n or a PDMS-based gel or PDMS-based elastomer. Non-limiting examples of PDMS polymers include those sold under the trademark Sylgard®, and particularly Sylgard® 182, Sylgard® 184, Sylgard® 186 and Sylgard® 527. In certain embodiments, the silicone phase of the silicone-based biophotonic composition can be prepared by using commercial kits such as Sylgard® 184 Silicone Elastomer kit. The kit consists in two-part liquid components, the base (part A) and the curing agent or catalyst (part B), both based on polydimethylsiloxane. When mixed at a ratio of l(A)/l(B), the mixture cures to a flexible and transparent elastomer.

In other embodiments, the chromofilm may be prepared in a manner to provide for tunable flexibility were desired.

In some embodiments, the chromofilm has a transmittance that is more than about 20%, 30%, 40%, 50%, 60%, 70%, or 75% within the visible range. In some embodiments, the transmittance exceeds 40%, 41%, 42%, 43%, 44%, or 45% within the visible range. In some embodiments, the silicone-based biophotonic composition has a light transmittance of about 40-100%, 45-100%, 50-100%, 55-100%, 60-100%, 65-100%, 70-100%, 75-100%, 80-100%, 85-100%, 90-100%, or 95-100%.

The chromofilm of the present disclosure may be deformable. They may be elastic or non-elastic (i.e. flexible or rigid). The chromofilm, for example, may be in a peel-off form (‘peelable’) to provide ease and speed of use. In certain embodiments, the tear strength and/or tensile strength of the peel-off form is greater than its adhesion strength. This may help handleability of the chromofilm. It will be recognized by one of skill in the art that the properties of the peel-off chromofilm such as cohesiveness, flexibility, elasticity, tensile strength, and tearing strength, can be determined and/or adjusted by methods known in the art such as by selecting suitable PDMS-based compositions and adapting their relative ratios.

The chromofilm may be provided in a pre-formed shape. In certain embodiments, the pre-formed shape is in the form of, including, but not limited to, a film. In certain embodiments, the pre-formed shape is in the form of a body part such as for example, a face or a portion thereof, a leg or a portion thereof, an arm or a portion thereof. In certain embodiments, the pre-formed shape is a face mask, a patch, a dressing, or bandage. In certain embodiments, the pre-formed shapes can be customized for the individual user by trimming to size. In certain embodiments, perforations are provided around the perimeter of the pre-formed shape to facilitate trimming.

A chromofilm of the disclosure can be configured with a shape and/or size for application to a desired portion of a subject's body. For example, the chromofilm can be shaped and sized to correspond with a desired portion of the body to receive the biophotonic treatment. Such a desired portion of skin can be selected from, but not limited to, the group consisting of a skin, head, forehead, scalp, nose, cheeks, lips, ears, face, neck, shoulder, arm pit, arm, elbow, hand, finger, abdomen, chest, stomach, back, buttocks, sacrum, genitals, legs, knee, feet, toes, nails, hair, any boney prominences, and combinations thereof, and the like. Thus, the chromofilm of the disclosure can be shaped and sized to be applied to any portion of skin on a subject's body. For example, the chromofilm can be in the form of a sock, hat, glove or mitten shaped form.

In certain embodiments, the chromofilm is in the form of a wound dressing or a bandage. It may be used on a wound to prevent or limit scar formation, or on an existing scar to diminish the appearance of the scar.

In some embodiments, the chromofilm has a tensile strength that is at least about 50 kPa, at least about 100 kPa, at least about 200 kPa, at least about 300 kPa, at least about 400 kPa, at least about 500 kPa, at least about 600 kPa, at least about 700 kPa, at least about 800 kPa, at least about 900 kPa, at least about 1 MPa, at least about 2 MPa or at least about 3 MPa, or at least about 5 MPa, or at least about 6 MPa. In some embodiments, the tensile strength of the silicone-based biophotonic composition is up to about 10 MPa.

In some embodiments, the chromofilm has a tear strength of from about 0.1 N/mm to about 5 N/mm. In some embodiments, the tear strength is from about 0.1 N/mm to about 0.5 N/mm, from about 0.25 N/mm to about 0.75 N/mm, from about 0.5 N/mm to about 1.0 N/mm, from about 0.75 N/mm to about 1.25 N/mm, from 5 about 1.0 N/mm to about 1.5 N/mm, from about 1.5 N/mm to about 2.0 N/mm, from about 2.0 N/mm to about 2.5 N/mm, from about 2.5 N/mm to about 3.0 N/mm, from about 3.0 N/mm to about 3.5 N/mm, from about 3.5 N/mm to about 4.0 N/mm, from about 4.0 N/mm to about 4.5 N/mm, from about 4.5 N/mm to about 5.0 N/mm.

In some embodiments, the chromofilm has adhesion strength of from about 0.01 N/mm to about 0.60 N/mm. In some embodiments, the adhesion strength is from about 0.20 N/mm to about 0.40 N/mm, or from about 0.25 N/mm to about 0.35 N/mm. In some embodiments, the adhesion strength is less than about 0.10 N/mm, less than about 0.15 N/mm, less than about 0.20 N/mm, less than about 0.25 N/mm, less than about 0.30 N/mm, less than about 0.35 N/mm, less than about 0.40 N/mm, less than about 0.45 N/mm, less than about 0.55 N/mm or less than about 0.60 N/mm.

ii) Light-Emitting Support

In some embodiments, the light-emitting support of the present technology comprises a substantially flat support and a light-emitting system. In some implementations, the substrate is a fiber substrate comprising a plurality of light-emitting elements. In some instances, the light-emitting elements are LEDs lights. In some other implementations, the light-emitting elements are quantum dots.

In some implementations, the LEDs are fixed onto preformed textile substrate. Existing LED technology such as surface mount display chip LEDs are suitable as they have a low profile and are very small therefore do not affect the handle of the fabric once attached. The chip LEDs are placed onto a fabric substrate with at least two electrically conductive textile tracks and fixed with electrically conductive adhesive such as silver filled silicone adhesive or silver filled epoxy resin. The two electrically conductive textile tracks have been preformed so as to be dimensionally suitable to make contact with the ends of the SMD chip LED when it is placed on to them. The electrically conductive textile tracks may be a woven, non-woven, knitted, stitched series of electrically conductive fibers or yarns incorporated into the textile structure or a series of electrically conductive tracks printed onto a textile substrate.

Many LEDs may be placed on the textile substrate to form a row, array or matrix of LEDs. Using electronic matrix drivers (common in LCD or LED display circuits) each LED can be driven separately. Once all the LEDs are fixed in place on the fabric they are covered with an encapsulant layer to provide durability to textile end-uses. This encapsulant layer may be a textile silicone sealant, or a fabric, or a polymer coating. It is important that the encapsulant layer is flexible so as to maintain the flexible properties of the textile fabric.

The electrically conductive textile tracks may be positioned within the textile so as to conform to a specific width and spacing dimension. The LEDs may be positioned onto the textile member by hand or using modified “pick and place” techniques known to those in the electronics assembly industry.

In some embodiments, the light-emitting elements can be arranged on the support in various ways, with regular and irregular arrangements being possible. The light-emitting elements may be arranged in a regular grid or in a manner to achieve a homogeneous light emission distribution by supplying each light-emitting element substantially with the same light intensity. On the other hand, the light-emitting elements may be arranged for generating predefined homogeneous light emission distributions. In this context, the light-emitting elements may be distributed homogeneously on the support (e.g., in rows and columns/grid). Therefore, a desired light emission distribution can be achieved by expressing it as a function of two coordinates, which substantially correspond to the above mentioned row numbers and column numbers, respectively; and subsequently feeding the optical fibers associated to individual light-emitting elements with light of an intensity that corresponds to the function values.

There are various possibilities for fixing the light-emitting elements to the support. In some instances, the light-emitting elements are stitched to the support. In this way, it is possible to achieve a well-defined arrangement of the individual light-emitting elements at the intended positions on the support while nonetheless having a rather loose arrangement of the supplying optical fibers. The result is a light-emitting textile structure having great flexibility.

In some embodiments, the substrate has a light reflecting backside that allows an increased light emission in the opposite front direction. In the present context, an optically effective layer may be any type of material sheet that exerts a predefined influence on the emission characteristics of the substrate. In particular, said sheet may act as diffuser. In some instances, the substrate is provided both with a light-reflecting backside and with an optically effective layer arranged at the front side thereof.

The light-emitting support is designed for medical purposes, in particular for biophotonic therapy or treatment or for photobiomodulation therapy or treatment.

ii) Method of Uses

In some embodiments, the present disclosure provides a method for using the biophotonic system of the present disclosure. In some implementations of these embodiments, the method comprises applying the chromofilm of the biophotonic systems of the present disclosure to the area of the skin or tissue in need of biophotonic treatment.

The method further comprises applying the light-emitting support in an operational configuration with the chromofilm. In some instances, the light-emitting support is placed on top of the chromofilm.

The light-emitting support is then activated (e.g., powered) so as to activate the plurality of light-emitting elements and to cause them to illuminate the chromofilm, particularly to illuminate the light-absorbing agents in the chromofilm. In some implementations, the light-emitting support having a wavelength that overlaps with an absorption spectrum of the light-absorbing agents of the chromofilm. The light-emitting elements are illuminated for a suitable illumination period depending on the type of biophotonic treatment that is being performed.

In some implementations of these embodiments, following the illumination of the chromofilm, the light-emitting support is removed from the chromofilm and the chromofilm is kept on the area of the skin or tissue treated to promote further treatment.

The biophotonic systems of the present disclosure may have cosmetic and/or medical benefits. They may be used to promote skin rejuvenation and skin conditioning, or to promote the treatment of a skin disorder such as acne, eczema, dermatitis or psoriasis, or to promote tissue repair, modulate inflammation, modulate collagen synthesis, reduce or avoid scarring, or promote wound healing including reducing depth of periodontitis pockets. In certain embodiments, the biophotonic systems of the disclosure may be used to treat acute inflammation, which may present itself as pain, heat, redness, swelling and loss of function, and which includes those seen in allergic reactions such as insect bites e.g.; mosquito, bees, wasps, poison ivy, or post-ablative treatment.

Accordingly, in certain embodiments, the present disclosure provides a method for treating acute inflammation. In certain embodiments, the present disclosure provides a method for providing skin rejuvenation or for improving skin condition, treating a skin disorder, preventing or treating scarring, and/or accelerating wound healing and/or tissue repair.

In the methods of the present disclosure, the primary characteristic of suitable light-emitting elements will be that they emit light in a wavelength (or wavelengths) appropriate for activating the one or more light-absorbing agents present in the chromofilm. In one embodiment, the light emitted by the light-emitting elements have a wavelength between about 200 to 800 nm, or between about 400 and 600 nm, or between about 400 and 700 nm. In yet another embodiment, the light emitted from the light-emitting elements is blue light, or green light, or yellow light, or orange light, or red light, or a combination thereof. Suitable power densities for non-collimated light sources (LED, halogen or plasma lamps) are in the range from about 0.1 mW/cm² to about 200 mW/cm². Suitable power density for laser light sources are in the range from about 0.5 mW/cm² to about 0.8 mW/cm². In some embodiments of the methods of the present disclosure, the light has an energy at the subject's skin surface of between about 0.1 mW/cm² and about 500 mW/cm², or 0.1-300 mW/cm², or 0.1-200 mW/cm², wherein the energy applied depends at least on the condition being treated, the wavelength of the light, the distance of the skin from the light source and the thickness of the biophotonic material. In certain embodiments, the light at the subject's skin is between about 1-40 mW/cm², or 20-60 mW/cm², or 40-80 mW/cm², or 60-100 mW/cm², or 80-120 mW/cm², or 100-140 mW/cm², or 30-180 mW/cm², or 120-160 mW/cm², or 140-180 mW/cm², or 160-200 mW/cm², or 110-240 mW/cm², or 110-150 mW/cm², or 190-240 mW/cm².

The activation of the light-absorbing agents in the chromofilm may take place almost immediately on illumination (femto- or pico seconds). A prolonged exposure period may be beneficial to exploit the synergistic effects of the absorbed, reflected and reemitted light of the biophotonic systems of the present disclosure and its interaction with the tissue being treated. In one embodiment, the time of exposure of the tissue or skin or biophotonic system to actinic light is a period between 0.01 minutes and 90 minutes. In another embodiment, the time of exposure of the tissue or skin or chromofilm to actinic light is a period between 1 minute and 5 minutes. In some other embodiments, the chromofilm is illuminated for a period between 1 minute and 3 minutes. In certain embodiments, light is applied for a period of 1-30 seconds, 15-45 seconds, 30-60 seconds, 0.75-1.5 minutes, 1-2 minutes, 1.5-2.5 minutes, 2-3 minutes, 2.5-3.5 minutes, 3-4 minutes, 3.5-4.5 minutes, 4-5 minutes, 5-10 minutes, 10-15 minutes, 15-20 minutes, or 20-30 minutes. In certain embodiments, the chromofilm may be re-illuminated at certain intervals.

In certain embodiments of the method of the present disclosure, the biophotonic system may be applied to the tissue, such as on the face, once, twice, three times, four times, five times or six times a week, daily, or at any other frequency. The total treatment time may be one week, two weeks, three weeks, four weeks, five weeks, six weeks, seven weeks, eight weeks, nine weeks, ten weeks, eleven weeks, twelve weeks, or any other length of time deemed appropriate. In certain embodiments, the total tissue area to be treated may be split into separate areas (cheeks, forehead), and each area treated separately. For example, the silicone-based biophotonic composition may be applied topically to a first portion, and that portion illuminated with light, and the composition then removed. Then a biophotonic system is applied to a second portion, illuminated and removed. Finally, the silicone-based biophotonic composition is applied to a third portion, illuminated and removed.

In certain embodiments, the biophotonic system of the present disclosure may be used to treat to burns.

In certain embodiments, the biophotonic system of the present disclosure may be used to effect early burns conversion, and without being bound by any theory, where such burns are due to excess inflammation, tissue ischemia, and cell autophagy, where such burns conversion occurring within 24 hours of the initial burn.

In certain embodiments, the biophotonic system of the present disclosure may be used to treat burns attributed to combat related injuries, suffered in blast injuries, and exposure to chemical and/or radiation type burns.

In certain embodiments, the biophotonic system of the present disclosure may be applied to the tissue immediately at the point after the subject had been exposed to a traumatic event, including combat related trauma, ex. exposure to fire, or as a result of exposure to a blast, within a 24-hour period.

In certain embodiments, the biophotonic system may be used following wound closure to optimize scar revision. In this case, the biophotonic system may be applied at regular intervals such as once a week, or at an interval deemed appropriate by the physician. In certain embodiments, the biophotonic system may be used following ablative skin rejuvenation treatment to maintain the condition of the treated skin. In this case, the biophotonic system may be applied at regular intervals such as once a week, or at an interval deemed appropriate by the physician.

The biophotonic system of the present disclosure may be useful in promoting skin rejuvenation or improving skin condition and appearance.

The silicone-based biophotonic system of the present disclosure promotes skin rejuvenation. In certain embodiments, the biophotonic system may promote skin conditioning such as skin luminosity, reduction of pore size, reducing blotchiness, making even skin tone, reducing dryness, and tightening of the skin. In certain embodiments, the biophotonic system may promote collagen synthesis. In certain other embodiments, the biophotonic system may reduce, diminish, retard or even reverse one or more signs of skin aging including, but not limited to, appearance of fine lines or wrinkles, thin and transparent skin, loss of underlying fat (leading to hollowed cheeks and eye sockets as well as noticeable loss of firmness on the hands and neck), skin aging due bone loss (wherein bones shrink away from the skin due to bone loss, which causes sagging skin), dry skin (which might itch), inability to sweat sufficiently to cool the skin, unwanted facial hair, freckles, age spots, spider veins, rough and leathery skin, fine wrinkles that disappear when stretched, loose skin, or a blotchy complexion. In certain embodiments, the biophotonic system may induce a reduction in pore size, enhance sculpturing of skin subsections, and/or enhance skin translucence.

In some embodiments, the pore size allows exudate from the skin or the tissue treated to be evacuated from the chromofilm.

The biophotonic system of the present disclosure may be used in a treatment of a skin disorder that may include, but is not limited to, erythema, telangiectasia, actinic telangiectasia, basal cell carcinoma, contact dermatitis, dermatofibrosarcoma protuberans, genital warts, hidradenitis suppurativa, melanoma, merkel cell carcinoma, nummular dermatitis, molloscum contagiosum, psoriasis, psoriatic arthritis, rosacea, scabies, scalp psoriasis, sebaceous carcinoma, squamous cell carcinoma, seborrheic dermatitis, seborrheic keratosis, shingles, tinea versicolor, warts, skin cancer, pemphigus, sunburn, dermatitis, eczema, rashes, impetigo, lichen simplex chronicus, rhinophyma, perioral dermatitis, pseudofolliculitis barbae, erythema multiforme, erythema nodosum, granuloma annulare, actinic keratosis, purpura, alopecia areata, aphthous stomatitis, drug eruptions, dry skin, chapping, xerosis, ichthyosis vulgaris, fungal infections, herpes simplex, intertrigo, keloids, keratoses, milia, moluscum contagiosum, pityriasis rosea, pruritus, urticaria, and vascular tumors and malformations. Dermatitis includes contact dermatitis, atopic dermatitis, seborrheic dermatitis, nummular dermatitis, generalized exfoliative dermatitis, and statis dermatitis. Skin cancers include melanoma, basal cell carcinoma, and squamous cell carcinoma.

The biophotonic system of the present disclosure may be used to treat acne. As used herein, “acne” means a disorder of the skin caused by inflammation of skin glands or hair follicles. The biophotonic system of the disclosure can be used to treat acne at early pre-emergent stages or later stages where lesions from acne are visible. Mild, moderate and severe acne can be treated with embodiments of the silicone-based biophotonic compositions and methods.

The biophotonic system of the present disclosure may be used to treat various types of acne. Some types of acne include, for example, acne vulgaris, cystic acne, acne atrophica, bromide acne, chlorine acne, acne conglobata, acne cosmetica, acne detergicans, epidemic acne, acne estivalis, acne fulminans, halogen acne, acne indurata, iodide acne, acne keloid, acne mechanica, acne papulosa, pomade acne, premenstral acne, acne pustulosa, acne scorbutica, acne scrofulosorum, acne urticata, acne varioliformis, acne venenata, propionic acne, acne excoriee, gram negative acne, steroid acne, and nodulocystic acne.

The biophotonic system of the present disclosure may be used to treat wounds, promote wound healing, and promote tissue. Wounds that may be treated by the biophotonic system of the present disclosure include, for example, injuries to the skin and subcutaneous tissue initiated in different ways (e.g., pressure ulcers from extended bed rest, wounds induced by trauma or surgery, burns, blast and chemical burns, ulcers linked to diabetes or venous insufficiency, wounds induced by conditions such as periodontitis) and with varying characteristics. In certain embodiments, the present disclosure provides biophotonic systems for treating and/or promoting the healing of, for example, burns, incisions, excisions, lesions, lacerations, abrasions, puncture or penetrating wounds, surgical wounds, contusions, hematomas, crushing injuries, amputations, sores and ulcers.

Biophotonic systems of the present disclosure may be used to treat and/or promote the healing of chronic cutaneous ulcers or wounds, which are wounds that have failed to proceed through an orderly and timely series of events to produce a durable structural, functional, and cosmetic closure. The vast majority of chronic wounds can be classified into three categories based on their etiology: pressure ulcers, neuropathic (diabetic foot) ulcers and vascular (venous or arterial) ulcers. For example, the present disclosure biophotonic systems for treating and/or promoting healing of a diabetic ulcer. In other examples, the present disclosure provides biophotonic systems for treating and/or promoting healing of a pressure ulcer. Pressure ulcers include bed sores, decubitus ulcers and ischial tuberosity ulcers and can cause considerable pain and discomfort to a patient.

iv) Kits

The present disclosure also provides kits for preparing a biophotonic system and/or providing any of the components required for forming a biophotonic system of the present disclosure.

In some embodiments, the kit includes the components of the biophotonic system of the present disclosure. In some embodiments, the kit includes one or more chromofilm as defined herein; and one or more light-emitting support. In some instances, the kit may further comprise fastening means for fastening the chromofilm with the light-emitting support.

In certain embodiments of the kit, the kit may further comprise written instructions on how to use the biophotonic system in accordance with the present disclosure.

### EXAMPLE

### Example 1—Wearable Biophotonic System

A wearable light source is designed to be used within the biophotonic system of the present technology. The device a wearable band is designed that attaches to the patient's upper or lower limb and covers an area of approximately 25-100 cm². Uniform light intensity within the green and blue spectrum at a wavelength range of 450-565 nm is distributed with a surface variance of less than 50%. The device is operated without exceeding a maximum skin temperature of 45 degrees Celsius and is reliable to operate for 20 cycles of eight hours. Upon the completion of each cycle, the device is shut-off automatically and indicates to the patient that the treatment is complete. Powered by a rechargeable battery, the light source emits a similar energy density of 150-500 mJ/cm². The device is designed with a focus on treating chronic wounds found on diabetic patients with limited mobility and is flexible and comfortable enough for their use.

The device requires a power source, a light source, a band complete with Velcro to attach to the user and a microcontroller with peripherals to measure the treatment time, battery life and heat of the skin surface.

Variations and modifications will occur to those of skill in the art after reviewing this disclosure. The disclosed features may be implemented, in any combination and sub-combinations (including multiple dependent combinations and sub-combinations), with one or more other features described herein. The various features described or illustrated above, including any components thereof, may be combined or integrated in other systems. Moreover, certain features may be omitted or not implemented. Examples of changes, substitutions, and alterations are ascertainable by one skilled in the art and could be made without departing from the scope of the information disclosed herein.

All references cited herein are incorporated by reference in their entirety and made part of this application.

Practice of the disclosure will be still more fully understood from the following examples, which are presented herein for illustration only and should not be construed as limiting the disclosure in any way.

1. A biophotonic system comprising:
at least one chromofilm; and
a light-emitting support operably connectable to the chromofilm;
wherein operable connection of the light-emitting support and the at least one chromofilm allows for the light-emitting support to illuminate the at least one chromofilm.

2. The biophotonic system according to claim 1, wherein the light-emitting support is a light-emitting fabric.

3. The biophotonic system according to claim 1, wherein the light-emitting support is a light-emitting textile.

4. The biophotonic system according to claim 1, wherein the at least one chromofilm comprises a silicone-based substrate.

5. The biophotonic system according to claim 4, wherein the silicone-based substrate comprises light-absorbing agents.

6. The biophotonic system according to claim 1, wherein the light-absorbing agents are xanthene dyes.

7. The biophotonic system according to claim 6, wherein the xanthene dyes are selected from: eosin B, eosin Y, fluorescein, and Rose Bengal.

8. The biophotonic system according to claim 1, wherein the light-emitting support comprises a plurality of light-emitting elements.

9. The biophotonic system according to claim 8, wherein the plurality of light-emitting elements are LEDs.

10. The biophotonic system according to claim 8, wherein the plurality of light-emitting elements are quantum dots.

11. The biophotonic system according to claim 1, wherein the light-absorbing agents are encapsulated.

12. A method for biophotonic treatment of an area of a tissue, the method comprising:
a) applying a biophotonic system to an area of the tissue to be treated; the biophotonic system comprising at least one chromofilm and a light-emitting support operably connected to the chromofilm; and
b) illuminating the chromofilm with the light-emitting support.

13. A method for biophotonic treatment of an area of a tissue, the method comprising:
a) applying a chromofilm to the area of the tissue;
b) connecting a light-emitting support to the chromofilm; and
c) illuminating the chromofilm with the light-emitting support,
wherein the illumination of the chromofilm causes the chromofilm to emit fluorescence.

14. A kit for biophotonic treatment, the kit comprising the biophotonic system as defined in claim 1.

15. (canceled)
